(19) United States
(12) Patent Application Publication
(10) Pub. No.: US 2004/0241200 A1
(43) Pub. Date: Dec. 2, 2004

(54) Personal care products that do not contain tetramer and/or pentamer cyclomethicones

(76) Inventors: Daniel Winn, Kingston, NJ (US); Marvin Goldberg, Jackson, NJ (US); Lawrence B. Moss, Manalapan, NJ (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(21) Appl. No.: 10/848,088

(22) Filed: May 18, 2004

Related U.S. Application Data
(60) Provisional application No. 60/471,556, filed on May 19, 2003.

(51) Int. Cl. A61K 7/06; A61K 7/11

(52) U.S. Cl. 424/401; 424/70.11

(57) ABSTRACT

The invention provides personal care products that do not contain D4 and/or D5 cyclomethicones but which do have the skin feel, spreadability, and other physical and chemical characteristics of personal care products that do contain these cyclomethicones. The products of the invention include a neopentyl polyol polyester(s) derived from neopentyl glycol, an isoparaffin(s), and a personal care formulation. Also included are replacement compositions for use in personal care products (so that D4 and D5 cyclomethicones can be excluded from the formulation) and methods of preparing personal care products and replacement compositions.

### CROSS-REFERENCE TO RELATED APPLICATIONS

This application claims priority to U.S. Provisional Application Ser. No. 60/471,556, filed May 19, 2003, the contents of which are incorporated herein by reference.

### BACKGROUND OF THE INVENTION

Silicone fluids are widely used in toiletry cosmetics and other personal care products. Most frequently used are the dimethicones, the cyclomethicones, and phenyltrimethicone. Incorporated into a product, these silicone fluids provide a variety of benefits such as improved slip, reduction of tack, impartation of emolliency, and other modifications of the “feel” characteristics of the formulation. The lower viscosity cyclomethicone fluids have been considered to be particularly useful by personal care product formulators, as they tend to provide the aforementioned benefits without contributing to oiliness or greasiness, and give what many consider to be a “dry” skin feel after application. This attribute, as well as the others, is highly desirable in personal care products such as, for example, creams, lotions, antiperspirants, shaving products, and make up products. Additional benefits are that the lower viscosity cyclomethicone fluids, particularly the tetramer (D4) and the pentamer (D5) cyclomethicones are excellent dispersing and spreading agents, are generally water white in color, low in odor, have some degree of volatility, and are resistant to chemical and oxidative attack.

There has been recent concern regarding the safety of the use of silicone fluids on the skin. In light of potential links between D4 and D5 cyclomethicone and silicone degradation products to the development of autoimmune disorders in persons with silicone implants, such as silicone breast implants, these materials have recently come under higher scrutiny by the United States Food and Drug Administration. Although definitive links have not been confirmed, many personal care product formulators are taking steps to reduce and/or eliminate these ingredients from formulations. There is a need in the art to find alternative non-silicone fluids that provide the benefits of the D4 and D5 cyclomethicone fluids without the real or perceived risks.

Attempts have been made to replace these lower viscosity silicone fluids in personal care products with commonly used esters, such as isopropyl laurate, isononyl isononanoate and 2-ethylhexyl 2-ethylhexanoate. These esters provide some characteristics similar to the low viscosity silicone fluids, such as emolliency; however, because they are monoesters, they tend to be slower spreading, oilier feeling, and can tend to have a disagreeable odor.

Thus, there remains an unmet need in the art for a compound that can provide in a personal care product the physical and chemical characteristics contributed by the lower viscosity cyclomethicone fluids, particularly the tetramer and pentamer cyclomethicone fluids.

### BRIEF SUMMARY OF THE INVENTION

The invention provides a personal care product that may not contain lower viscosity cyclomethicone fluids, specifically, does not contain the tetramer and/or pentamer cyclomethicone fluids. The personal care product includes at least one neopentyl polyol polyester derived from neopentyl glycol, at least one isoparaffin, and a personal care formulation. This personal care product may have skin feel characteristics similar to those exhibited by a personal care composition containing a D4 and/or D5 cyclomethicone.

In addition, the invention described herein is a replacement composition to replace a lower viscosity cyclomethicone in a personal care composition. The replacement composition comprises at least one neopentyl polyol polyester derived from neopentyl glycol and at least one isoparaffin. The replacement composition has a kinematic viscosity of about 2 to about 6 centistokes at 25° C. and a vapor pressure of about 0.2 to about 3 mm Hg, when measured at 25° C. Moreover, the replacement composition does not contain a D4 and/or D5 cyclomethicone.

The invention also includes several methods. A method of preparing a replacement composition to replace lower viscosity cyclomethicone in a personal care product is provided. The method includes blending at least one neopentyl polyol polyester derived from neopentyl glycol and at least one isoparaffin. The resultant replacement composition has a kinematic viscosity of about 2 to about 6 centistokes at 25° C. and a vapor pressure of about 0.2 to about 3 mm Hg when measured at 25° C. Again, this replacement composition does not contain a D4 and/or D5 cyclomethicone.

Also included is a method of preparing personal care products that do not contain D4 and/or D5 cyclomethicone(s). The method includes preparing a composition by blending at least one neopentyl polyol polyester derived from neopentyl glycol and at least one paraffin. This composition has a kinematic viscosity of about 2 to about 6 centistokes at 25° C. and a vapor pressure of about 0.2 to about 3 mm Hg when measured at 25° C. In the practice of the method, this composition is then blended with a personal care formulation and the resultant personal care product does not contain a D4 and/or D5 cyclomethicone(s). The personal care formulation may be, for example, an antiperspirant personal care formulation, a hair conditioning personal care formulation, and/or a decorative cosmetic formulation. The method may also be slightly varied such that the personal care product is prepared by steps including blending at least one neopentyl polyol polyester derived from neopentyl glycol, at least one isoparaffin, and a personal care formulation, such that the personal care product does not contain a D4 and/or D5 cyclomethicone(s).

Finally, included within the scope of the invention is a method of preparing a personal care product including (a) preparing a composition by blending at least one neopentyl polyol polyester derived from neopentyl glycol and at least one isoparaffin. The composition has a kinematic viscosity of about 2 to about 6 centistokes at 25° C. and a vapor pressure of about 0.2 to about 3 mm Hg when measured at 25° C. and (b) blending the composition of (a) with a personal care formulation such that the personal care product does not contain a D4 and/or D5 cyclomethicone and has skin feel characteristics similar to a D4 and/or D5 cyclomethicone containing a personal care product.

### DETAILED DESCRIPTION OF THE INVENTION

It has been found that by blending neopentyl polyol polyesters with isoparaffins, the viscosity, spreading characteristics, volatility profile, and “skin feel” of cyclomethicone fluids can be matched, resulting in compositions that can be used as replacement compositions for the D4 and/or D5 cyclomethicone in personal care products. Using these compositions, one may prepare personal care products that, to the end user, feel, apply and perform like D4 and/or D5 cyclomethicone-containing products.

By “personal care product” it is meant any composition that is used or marketed as a material to be applied to the skin, hair, or nails, and/or the stratum corneum of human or animal subjects for cosmetic, aesthetic, and/or therapeutic effects, regardless of the delivery form of the composition, and may include phases or intermediate preparations ultimately formulated into personal care products. Such products can include grooming products, such as soaps, cleansers, shampoos, skin or hair conditioners, shaving creams, lotions, and gels; hair sprays, gels, tonics, mousses, pomades, and lacquers; antiperspirants and deodorants; skin lotions, creams, mousses, and ointments, and nail and cuticle care products, such as polishes and creams. Also included are color-imparting cosmetics (decorative cosmetics) such as lipsticks, mascaras, foundations, eye shadows and other decorative cosmetics.

The methods, replacement compositions, and personal care products of the invention all include use of at least one neopentyl polyol polyester that is derived from neopentyl glycol. Any one or more of the known neopentyl polyol polyesters derived from neopentyl polyols known or developed in the art are suitable for use in the products, replacement compositions, and methods of the invention. Preferred may be neopentyl glycol diesters (of short chain carboxylic acids [branched or unbranched]). Particularly preferred may be neopentyl polyol polyesters derived from neopentyl glycol, trimethylol propane, pentaerythritol, and dipentaerythritol with linear and/or branched carboxylic acids of, for example, five to ten carbon atoms.

Neopentyl glycols are preferred, especially if low viscosity and low odor is desired in the personal care product. For example, neopentyl glycol diheptanoate (NDH) is a water white, odorless liquid with a viscosity of about 7.5 centistokes at 25° C. NDH is a very fast spreading liquid with a very low volatility, and provides a soft, silky smooth, powdery after feel on the surface of the skin. It is particularly usefully when used as an emollient in creams, lotions, antiperspirants, and color cosmetics.

Also used in the personal care personal care products, replacement compositions, and methods of the invention are isoparaffins. Isoparaffins are produced from the hydrogenation of oligomeric products of butylene raffinate; any known or to be developed in the art may be used. Particularly preferred are isoparaffins having about ten to twenty-five carbon atoms, with those having about ten to fourteen carbon atoms being preferred. In addition, isoparaffins having ten, eleven, twelve, thirteen, fourteen, fifteen, sixteen, seventeen, or eighteen carbon atoms may be used. Mixtures of isoparaffins may be used, or only one may be selected. Isoparaffins exist in fluid form; the isoparaffins or mixtures of isoparaffins may be referred to as isoparaffin fluids.

A preferred isoparaffin is isododecane (ID), a mixture of isoparaffins having twelve carbon atoms consisting mainly of 2,2,4,6,6-pentamethyl hexane. It is a colorless and almost odorless liquid with a kinematic viscosity of 1.6 centistokes at 25° C., and a vapor pressure of 1.4 mm Hg at 25° C. Also preferred, may be isoparaffins having ten to thirteen carbon atoms (C₁₀-C₁₃ isoalkanes) available under the tradename SOLTROL® 130 isoparaffin solvent, available from Chevron Phillips Chemical Company LP, The Woodlands, Tex., United States of America. It is a liquid with a vapor pressure of 1.5 mm Hg at 38° C. and a viscosity of 1.55 centistokes at 38° C.

Also included in the personal care products, replacement compositions, and methods of the invention are personal care formulations. By “personal care formulations” it is meant any formulations known or to be developed in the art for personal care products. Exemplary formulations include those for soap (liquid, solid, foam, or mousse), cleansers, creams, lotions, ointments, suspensions, shampoos, deodorants, antiperspirants, conditioning products for hair, skin, and nails, and decorative cosmetics (i.e., those that impart color to the hair, skin or nails upon application). The personal care formulations used in the invention may include organic components, inorganic components; color active ingredients (e.g., pigments and dyes); therapeutic active ingredients (e.g., vitamins, alphahydroxy acids, cortical steroids, amino acids, collagen, retinoids, antimicrobial compounds), sunscreens and/or UV absorbing compounds, reflective compounds, oils such as castor oil and olive oil; film formers, high viscosity oils, high molecular weight esters, antiperspirant active ingredients, glycol solutions, water, alcohols, emulsifiers, gellants, emollients, water, polymers, hydrocarbons, conditioning agents, and/or aliphatic esters.

Exemplary pigments and/or dyes include, but are not limited to, titanium dioxide, iron oxides, zinc oxide, ultramarine blue, manganese violet, mica powder, barium lake, calcium lake, aluminum lake, FD&C yellow, FD&C green, FD&C blue, and FD&C red.

Suitable conditioning agents, especially for hair conditioning formulations, may include polyquaternium polymers, alkamidopropyldimethyl amines, alkyltrimonium chlorides, alkytrimonium methyl sulfates, and ester quaternaries.

Exemplary high molecular weight esters include pentaerythritol, tetraisostearate (such as CRODAMOL® PTIS, Croda Corporation, Parsipanny, N.J., United States of America) and dipentaerythritol hexa C₅-C₉ acid esters (such as LEXFEEL® 350, Inolex Chemical Company, Philadelphia, Pa., United States of America). In some cases, such as in a formulation for a personal care product that is a decorative cosmetic, it may be desirable to disperse the color active ingredients (e.g., opaque or translucent pigments or dyes) in these high molecular weight esters and/or high viscosity oils, such as castor oil.

Typical film formers that can be used in the personal care formulation components of the invention include acrylate copolymers, such as AVALURE® AC120, Noveon Corporation, Cleveland, Ohio, United States of America), tricontanyl PVP (such as GANEX® WP-660, ISP Corporation, Wayne, N.J., United States of America), alpha-olefin/isopropyl maleate copolymer (such as PROFORMA® V1608, New Phase Technologies, Sugarland, Tex., United States of America). In general, film formers will be included in the formulation when preparing a product intended to smooth out surface irregularities in the stratum corneum and to provide a stable, immoveable matrix for any color active ingredients to be included.

The personal care formulation may be an antiperspirant including inorganic salts and/or inorganic salt/glycine complexes. Examples of antiperspirant active ingredients include, but are not limited to, activated aluminum chlorohydrate (“AACH”), aluminum zirconium polychlorhydrex/glycine complex (“ZAG”) or activated ZAG (“AZAG”). The antiperspirant active ingredient may be provided to the formulation in any form, including a powder, an aqueous solution, or a glycol solution (e.g., propylene glycol, 1,3-butylene glycol, and dipropylene glycol).

Conventional antiperspirant gels are microemulsions made up of two immiscible phases in which the particle size of the internal phase is typically less than 100 nanometers. In conventional practice, the formulation of clear antiperspirant gels will usually include the combination of an active ingredient phase and a vehicle phase. The active ingredient phase will normally include one or more of the previously mentioned antiperspirant active ingredients, either dissolved or pre-dissolved in water, propylene glycol, other glycols, or mixtures thereof. The vehicle phase will typically include an emulsifier/gellant and a mixture of oily ingredients that are meant to ease the spreading the antiperspirant on to the skin, and to reduce the feeling of drag, greasiness and/or tackiness. Common oily ingredients that may be used in the vehicle phase of these non-silicone personal care products may be hydrocarbons and aliphatic esters.

In the practice of the invention (regardless of the product or composition formed), the neopentyl polyol polyester(s) are blended with isoparaffin(s) to duplicate the spreading rate, viscosity, volatility, and/or skin feel of the silicone fluids. In this way, a replacement composition is provided. This blending may be accomplished by any means known or to be developed in the art including mixing, paddle mixing, homogenizing, shaking, stirring, or agitating. In general, the selected neopentyl polyol polyesters and isoparaffins may be present in the replacement composition and/or personal care products in any amount. However, it is generally preferred that the weight ratio of the at least one neopentyl polyol polyester to the at least one isoparaffin present in the composition is about eight parts to about five parts neopentyl polyol polyester to about two parts to five parts of isoparaffin (about 8:2 to about 5:5).

In the examples included herein, kinematic viscosity was tested at 25° C. using the ASTM (American Society of Testing and Materials, Conshohocken, Pa., United States of America) official method number D-445-97 (1997), the contents of which are incorporated herein by reference. Volatility was measured by determining the vapor pressure that is estimated using quantitative structure/activity relationship (QSAR) software provided by the Environmental Protection Agency (EPA). To test the spreading characteristics, a four inch WHATMAN® filter paper was positioned horizontally over an eight ounce jar. Fifty microliters of each product were pipetted into the center of the filter paper. The spreading area of the liquid was then measured at intervals of one, three, and five minutes. Faster spreading products will have a higher spreading area at each time interval.

The names for the components given in the replacement composition and personal care product examples are those used by the Cosmetic, Toiletry, and Fragrance Association (“CTFA”), as set forth in the CTFA International Cosmetic Ingredient Dictionary and Handbook, 9^{th} ed. (2002).

### EXAMPLE 1

### Preparation of a Replacement Composition to Replace a Lower Viscosity Cyclomethicone in a Personal Care Composition

A replacement composition was prepared using NDH and isododecane. It was designed to match the performance characteristics of a D4 cyclomethicone fluid in a personal care product. The replacement composition was prepared by mixing 30.0 g of NDH with 70.0 g of isododecane. The physical properties of the replacement composition as compared to a D4 fluid are shown in Table 1.

TABLE 1
Comparison of the properties of the replacement composition of the
invention to a D4 fluid:

| | Replacement | |
|---|---|---|
| Property | Composition | D4 Fluid |
| Kinematic viscosity (25° C.), cSt | 2.4 | 2.4 |
| Spreadability (1 minute), cm | 3.3 | 3.1 |
| Spreadability (3 minutes), cm | 3.7 | 3.5 |
| Spreadability (5 minutes), cm | 3.8 | 3.6 |
| Vapor pressure (25° C.), mm Hg | 1.1 | 1.0 |
| Vapor pressure (50° C.), mm Hg | 5.0 | 5.7 |
| Vapor pressure (100° C.), mm Hg | 56 | 60 |

As can be seen from the data shown in Table 1, the physical characteristics (kinematic viscosity, spreadability, and vapor pressure) of the replacement composition are almost identical to those of the D4 fluid. Thus, using this replacement composition, a personal care product can be formulated that has performance characteristics similar to those of a personal care product that contains a D4 fluid.

### EXAMPLE 2

### Preparation of a Replacement Composition to Replace a Lower Viscosity Cyclomethicone in a Personal Care Composition

A composition was prepared using NDH and isododecane. It was designed to match the performance characteristics of a D5 cyclomethicone fluid. The replacement composition was prepared by mixing 65.0 g of NDH with 35.0 g of isododecane. The properties of the replacement composition as compared to D5 cyclomethicone fluid are shown in Table 2.

TABLE 2
Comparison of the properties of the replacement composition of the
invention to a D5 cyclomethicone fluid:

| | Replacement | |
|---|---|---|
| Property | Composition | D4 Fluid |
| Kinematic viscosity (25° C.), cSt | 4.0 | 4.0 |
| Spreadability (1 minute), cm | 3.1 | 2.9. |
| Spreadability (3 minutes), cm | 3.5 | 3.4 |
| Spreadability (5 minutes), cm | 3.7 | 3.6 |
| Vapor pressure (25° C.), mm Hg | 0.9 | 0.5 |
| Vapor pressure (50° C.), mm Hg | 2.9 | 1.4 |
| Vapor pressure (100° C.), mm Hg | 30 | 20 |

As can be seen from the data shown in Table 2, the physical characteristics (kinematic viscosity, spreadability, and vapor pressure) of the replacement composition are almost identical to those of the D5 fluid. Thus, using this replacement composition, a personal care product can be formulated that has performance characteristics similar to those of a personal care product that contains a D5 fluid.

### EXAMPLE 3

### Preparation of a Replacement Composition to Replace a Lower Viscosity Cyclomethicone in a Personal Care Composition

A replacement composition was prepared using NDH and C₁₀-C₁₃ isoalkanes to match the performance characteristics of a D5 cyclomethicone fluid. 57.0 g of NDH were blended with 43.0 g of C₁₀-C₁₃ isoalkanes. Table 3 lists the properties of the blends as compared to the D5 cyclomethicone fluid.

TABLE 3
Comparison of the replacement composition
to D5 cyclomethicone fluid:

| | Replacement | |
|---|---|---|
| Property | Composition | D4 Fluid |
| Kinematic viscosity (25° C.), cSt | 4.0 | 4.0 |
| Spreadability (1 minute), cm | 3.0 | 2.9 |
| Spreadability (3 minutes), cm | 3.6 | 3.4 |
| Spreadability (5 minutes), cm | 3.8 | 3.6 |
| Vapor pressure (25° C.), mm Hg | 0.7 | 0.5 |
| Vapor pressure (50° C.), mm Hg | 3.4 | 1.4 |
| Vapor pressure (100° C.), mm Hg | 37.2 | 20 |

As can be seen from the data shown in Table 3, the physical characteristics (kinematic viscosity, spreadability, and vapor pressure) of the replacement composition are almost identical to those of the D5 fluid. Thus, using this replacement composition, a personal care product can be formulated that has performance characteristics similar to those of a personal care product that contains a D5 fluid.

### EXAMPLE 4

### Preparation of a Personal Care Product That is an Antiperspirant Stick That Results in Minimal Whitening of Skin Surface and Clothing and Does Not Contain D4 and/or D5 Cyclomethicones

Hydroxystearic acid 10 g and the replacement composition 56 g of Example 1 were combined in a vessel and warmed to about 80°-85° C. under gentle agitation until a clear solution is obtained. Once the solution was clear, it was allowed to cool to about 70° C. Subsequently, talc 6 g, aluminum zirconium tetrachlorohydrex/glycine 2.4 g and amorphous fumed silica 4 g are added. The mixture was agitated until uniformly mixed, then cooled to about 55-60° C. It was subsequently poured into canisters.

### EXAMPLE 5

### A Personal Care Product That is a Lotion for the Face and Does Not Contain a D4 and/or D5 Cyclomethicone

This facial lotion provides a dry, silky feel and a silicone-like aesthetic to the skin. Deionized water 75.6 g, glycerin 3.0 g, propylene glycol NF 2 g, and C₁₀-C₂₀ alkylacrylate cross polymer (PEMULEN TR-1, available from Noveon Corporation, Cleveland, Ohio, United States of America) 0.15 g were combined in the vessel under agitation until the cross polymer is dispersed. The mixture was warmed to about 70-75° C. Methylparaben 0.2 g and propylparaben 0.15 g were added.

The mixture was agitated until methylparaben and the propylparaben were completely dissolved. PEG-10 olive glycerides 0.50 g were added. Glyceryl stearate (and) PEG-100 stearate 3 g, cetyl alcohol (and) behenyl alcohol (and) behenic acid (and) stearyl alcohol (and) behenamidopropyl PG dimonium chloride 3.5 g, glyceryl dilaurate 0.5 g, vitamin E acetate 0.1 g, D-panthenol 0.3 g, and the replacement composition 11 g of Example 2 were combined in a separate vessel and warmed to about 70-75° C. with gentle agitation until a uniform dispersion was obtained. The contents of the first vessel were added to the second, and the resultant mixture was allowed to cool to about 60-65° C. The pH of the mixture was determined, and adjusted to about 6.0 to about 6.5 with triethanolamine. Finally, the mixture was poured into containers and allowed to cool to room temperature.

### EXAMPLE 6

### A Hair Conditioner Personal Care Product That Does Not Contain a D4 and/or a D5 Cyclomethicone

The hair conditioner personal care product results in hair having moisture, shine, and gloss. Deionized water 78 g, methylparaben 0.2 g, and propylparaben 0.15 g were combined in a vessel with agitation and warmed to about 70-75° C. until a clear solution was obtained. L-glutaminic acid 0.6 g was then added; agitation was continued until it was dissolved. In a separate vessel, also with agitation, cetearyl alcohol 7.5 g, stearamidopropyl dimethylamine 3.5 g, and the replacement composition of Example 2 were combined and heated to about 70-75° C. under agitation until a clear solution was obtained. The contents of the first vessel were added to the second, and the resultant mixture was allowed to cool slowly to about 20-25° C.

An emulsion was permitted to completely form. The pH of the emulsion was then determined and adjusted to about 5.0 to about 5.2 with citric acid. The resulting mixture was poured into containers.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A personal care product comprising:
(a) at least one neopentyl polyol polyester derived from neopentyl glycol,
(b) at least one isoparaffin, and
(c) a personal care formulation,
but which does not contain a tetramer and/or pentamer cyclomethicone.

2. The personal care product of claim 1, wherein the at least one neopentyl polyol polyester is independently a neopentyl glycol polyester derived from carboxylic acids.

3. The personal care product of claim 2, wherein the carboxylic acids have five to ten carbon atoms.

4. The personal care product of claim 1, wherein one of the at least one neopentyl polyol polyesters is neopentyl glycol diheptanoate.

5. The personal care product of claim 1, wherein the at least one isoparaffin independently has ten to fourteen carbon atoms.

6. The personal care product of claim 1, wherein one of the at least one isoparaffins is isododecane.

7. The personal care product of claim 1, wherein weight ratio of the at least one neopentyl polyol polyester to the at least one isoparaffin present in the composition is about 8 parts to about 5 parts neopentyl polyol polyester to about 2 parts to 5 parts isoparaffin.

8. The personal care product of claim 1, wherein the personal care formulation is selected from the group consisting of an antiperspirant personal care formulation, a hair conditioning personal care formulation, and a decorative cosmetic formulation.

9. A personal care product comprising:
(a) at least one neopentyl polyol polyester derived from neopentyl glycol,
(b) at least one isoparaffin, and
(c) a personal care formulation,
but which does not contain a tetramer and/or pentamer cyclomethicone and wherein the personal care product has a performance characteristic similar to a personal care composition that contains a tetramer and/or pentamer cyclomethicone.

10. A replacement composition to replace a lower viscosity cyclomethicone in a personal care composition, the replacement composition comprising at least one neopentyl polyol polyester derived from neopentyl glycol and at least one isoparaffin and having a kinematic viscosity of about 2 to about 6 centistokes at 25° C. and a vapor pressure of about 0.2 to about 3 mm Hg when measured at 25° C., and wherein the replacement composition does not contain a tetramer and/or pentamer cyclomethicone.

11. A method of preparing a replacement composition to replace a lower viscosity cyclomethicone in a personal care product, the method comprising blending at least one neopentyl polyol polyester derived from neopentyl glycol and at least one isoparaffin, wherein the resulting replacement composition has a kinematic viscosity of about 2 to about 6 centistokes at 25° C. and a vapor pressure of about 0.2 to about 3 mm Hg when measured at 25° C., and wherein the replacement composition does not contain a tetramer and/or pentamer cyclomethicone.

12. A method of preparing a personal care product comprising:
(a) preparing a composition by blending at least one neopentyl polyol polyester derived from neopentyl glycol and at least one isoparaffin, wherein the composition has a kinematic viscosity of about 2 to about 6 centistokes at 25° C. and a vapor pressure of about 0.2 to about 3 mm Hg when measured at 25° C.; and
(b) blending the composition of (a) with a personal care formulation,
wherein the personal care product does not contain a tetramer and/or pentamer cyclomethicone.

13. The method of claim 12, wherein the at least one neopentyl polyol polyester is independently a neopentyl glycol polyester derived from carboxylic acids.

14. The method of claim 13, wherein the carboxylic acids have five to ten carbon atoms.

15. The method of claim 12, wherein one of the at least one neopentyl polyol polyesters is neopentyl glycol diheptanoate.

16. The method of claim 12, wherein the at least one isoparaffin independently has ten to fourteen carbon atoms.

17. The method of claim 12, wherein one of the at least one isoparaffins is isododecane.

18. The method of claim 12, wherein weight ratio of the at least one neopentyl polyol polyester to the at least one isoparaffin present in the composition is about 8 parts to about 5 parts neopentyl polyol polyester to about 2 parts to 5 parts isoparaffin.

19. The method of claim 12, wherein the personal care formulation is selected from the group consisting of an antiperspirant personal care formulation, a hair conditioning personal care formulation, and a decorative cosmetic formulation.

20. A method of preparing a personal care product comprising blending at least one neopentyl polyol polyester derived from neopentyl glycol, at least one isoparaffin, and a personal care formulation, wherein the personal care product does not contain a tetramer and/or a pentamer cyclomethicone.

21. A method of preparing a personal care product comprising:
(a) preparing a composition by blending at least one neopentyl polyol polyester derived from neopentyl glycol and at least one isoparaffin, wherein the composition has a kinematic viscosity of about 2 to about 6 centistokes at 25° C. and a vapor pressure of about 0.2 to about 3 mm Hg when measured at 25° C.; and
(b) blending the composition of (a) with a personal care formulation,
but which does not contain a tetramer and/or pentamer cyclomethicone and wherein the personal care product has a performance characteristic similar to a personal care composition that contains a tetramer and/or pentamer cyclomethicone.

22. The method of claim 21, wherein the at least one neopentyl polyol polyester is independently a neopentyl glycol polyester derived from carboxylic acids.

23. The method of claim 22, wherein the carboxylic acids have five to ten carbon atoms.

24. The method of claim 21, wherein one of the at least one neopentyl polyol polyesters is neopentyl glycol diheptanoate.

25. The method of claim 21, wherein the at least one isoparaffin independently has ten to fourteen carbon atoms.

26. The method of claim 21, wherein one of the at least one isoparaffins is isododecane.

27. The method of claim 21, wherein weight ratio of the at least one neopentyl polyol polyester to the at least one isoparaffin present in the composition is about 8 parts to about 5 parts neopentyl polyol polyester to about 2 parts to 5 parts isoparaffin.

28. The method of claim 21, wherein the personal care formulation is selected from the group consisting of an antiperspirant personal care formulation, a hair conditioning personal care formulation, and a decorative cosmetic formulation.

29. A method of preparing a personal care product comprising blending at least one neopentyl polyol polyester derived from neopentyl glycol, at least one isoparaffin, and a personal care formulation, wherein the personal care product does not contain a tetramer and/or a pentamer cyclomethicone.
